(19) United States
(12) Patent Application Publication
(10) Pub. No.: US 2013/0261020 A1
(43) Pub. Date: Oct. 3, 2013

(54) Method of Diagnosing Down's Syndrome

(71) Applicant: University of Plymouth Enterprise Limited, Plymouth (GB)

(72) Inventors: Tracey Elizabeth MADGETT, Devon (GB); Neil David AVENT, Devon (GB)

(21) Appl. No.: 13/902,708

(22) Filed: May 24, 2013

Related U.S. Application Data
(63) Continuation-in-part of application No. PCT/GB2011/052322, filed on Nov. 25, 2011.

(30) Foreign Application Priority Data
Nov. 26, 2010 (GB) 1020071.5

(51) Int. Cl. C12Q 1/68

(52) U.S. Cl. CPC C12Q 1/6883
USPC 506/9; 436/501; 435/6.11; 435/7.92

(57) ABSTRACT

A method of diagnosing Down's syndrome, the method comprising identifying a different expression pattern of at least one diagnostic marker in a blood, plasma or serum sample from a patient compared to the normal expression pattern of the marker.

### PRIOR RELATED APPLICATIONS

This invention claims priority to and is a CIP application of PCT/GB2011/052322, filed on Nov. 25, 2011, and also claims priority to GB 1020071.5 filed Nov. 26, 2010. Each of these references is incorporated by reference in its entirety herein.

### FEDERALLY SPONSORED RESEARCH STATEMENT

Not applicable.

### FIELD OF THE INVENTION

This invention relates to the diagnosis of Down's syndrome, in particular, diagnostic markers for the non-invasive prenatal diagnosis of Down's syndrome.

### BACKGROUND

Down's syndrome is a relatively common chromosomal abnormality that causes mental retardation. It results from an extra copy (of which there are normally 2) of chromosome 21. The condition is referred to as trisomy 21 because of the three copies. All pregnancies are assessed for their potential risk for chromosomal abnormality (aneuploidy) by a series of screening tests for maternal plasma proteins that are fetally-derived via the placenta. Down's syndrome increases in incidence with increasing maternal age, and is presently screened for by certain maternal serum biomarkers including alpha fetoprotein (AFP), beta human chorionic gonadotrophin (B-hCG) and pregnancy associated plasma protein A (PAPP-A).

Unfortunately these tests are not fully diagnostic, each individual test is at best able to predict 85% accurately if the fetus being carried has Down's syndrome, there is also a 5-10% false positive prediction rate. A high-risk score for any of these markers may then be followed up by assessment of nuchal translucency, and then by invasive prenatal diagnosis to sample fetal cells to enable either karyotyping or quantitative fluorescent PCR to assess chromosomal numbers. Furthermore, the test can be quite painful and stressful for the expecting mother, and these invasive procedures impart a small but significant risk of procedural related loss due to spontaneous miscarriage. Further stress is incurred waiting for lab results, which can take significant time.

Because of the above disadvantages, significant efforts have been expended in seeking non-invasive alternatives to Down syndrome diagnostics. In recent years, much emphasis has been placed on using free fetal DNA and more recently free fetal mRNA found in maternal plasma. Despite these advances, a protein-based assay with greater screening and perhaps even diagnostic potential would provide a cheap and technically non-challenging alternative to the use of fetal derived nucleic acids.

There is, therefore, a need to provide diagnostic markers for Down's syndrome that can be detected non-invasively.

### SUMMARY OF THE INVENTION

According to a first aspect of the invention, there is provided a method of diagnosing Down's syndrome, the method comprising identifying a different expression pattern of at least one diagnostic marker in the blood, plasma or serum of a patient compared to the normal expression pattern of the marker, characterized in that the diagnostic marker is selected from those shown in Table 1 (or Tables 3 and 4).

TABLE 1
DOWN'S SYNDROME MARKERS

| Marker | |
|---|---|
| Number | Marker name |
| 1 | taste receptor type 1 member 1 |
| 2 | Zinc finger protein 704 |
| 3 | ATP synthase 0 subunit, mitochondrial precursor |
| 4 | l-acylglycerol-3-phosphate O-acyltransferase 3, isoform CRAb |
| 5 | Tripartite motif protein 46 |
| 6 | Myelin protein zero-like protein 2 precursor |
| 7 | ATP synthase coupling factor 6, mitochondrial precursor |
| 8 | 80 kDa MCM3-associated protein |
| 9 | Submaxillary gland androgen-regulated protein 3 homolog A |
| | precursor |
| 10 | Human cDNA clone |
| 11 | AP2-associated protein kinase 1 |
| 12 | Orphan nuclear receptor NR1D1 |
| 13 | Cystatin-B (Stefin-B) (Liver thiol proteinase inhibitor) |
| 14 | glial cell derived neurotrophic factor |
| 15 | Heat-shock protein beta-9 (HspB9). |
| 16 | MORC family CW-type zinc finger protein 3 |
| 17 | Serine protease inhibitor Kazal-type 7 precursor |
| 18 | Receptor-interacting serine/threonine-protein kinase 4 |
| 19 | Suppressor of cytokine signaling 1 |
| 20 | cDNA clone DKFZp434C2331 |
| 21 | Dual specificity protein phosphatase 15 |
| 22 | Trinucleotide repeat-containing protein 18 |
| 23 | Ubiquitin carboxyl-terminal hydrolase 16 |
| 24 | Paired mesoderm homeobox protein 2A |
| 25 | Serine/threonine-protein kinase 11 |
| 26 | Ribosome-binding protein 1 |
| 27 | Transcribed locus BX090181 |
| 28 | ATP synthase coupling factor 6, mitochondrial precursor |
| 29 | Zinc finger protein 488 |
| 30 | Uncharacterized protein C16orf3 |
| 31 | Pericentrin (Pericentrin B) (Kendrin). |
| 32 | E4F transcription factor 1 |
| 33 | v-ets erythroblastosis virus E26 oncogene homolog 2 (avian) |
| 34 | spire homolog 2 |
| 35 | Periodic tryptophan protein 2 homolog |
| 36 | Glycinamide ribonucleotide synthetase |
| 37 | HemK methyltransferase family member 2 |
| 38 | Trypsin-like serine protease |
| 39 | RRPI-like protein (Protein NNP-1) |
| 40 | Immunoglobulin heavy chain V gene segment |
| 41 | Coiled-coil-helix-coiled-coil-helix domain-containing protein 2 |
| 42 | DNA polymerase kappa |
| 43 | Inositol hexakisphosphate kinase 2 |
| 44 | Zinc finger protein 625 |
| 45 | leptin receptor |
| 46 | Blood vessel epicardial substance |
| 47 | Uncharacterized protein C13orf30 |
| 48 | Inhibitor of growth protein 5 |
| 49 | Zinc finger CCCH domain-containing protein 5. |
| 50 | Rhodopsin (Opsin-2). |
| 51 | Meiotic recombination protein DMC1/LIM15 homolog |
| 52 | CD lb molecule |
| 53 | Putative uncharacterized protein DKFZp761E198 |
| 54 | Ciliary dynein heavy chain 8 |

The method includes any one or more of the following embodiments, which can be combined in any combination:

A method of diagnosing Down's syndrome comprising identifying a different expression pattern of at least one diagnostic marker in a sample from a patient compared to the normal expression pattern of the marker, wherein the diagnostic marker is selected from those shown in Tables 1, 3 or 4.

A method of diagnosing Down's syndrome comprising identifying an increased amount of at least one diagnostic marker in a sample from a patient compared to the normal expression pattern of the marker, wherein the diagnostic marker is selected from marker number 1 to 39 inclusive of those shown in Table 1.

A method of diagnosing Down's syndrome, the method comprising identifying a decreased amount of at least one diagnostic marker in a sample from a patient compared to the normal expression pattern of the marker, wherein the diagnostic marker is selected from marker number 40 to 54 inclusive of those shown in Table 1.

A method of diagnosing Down's syndrome, the method comprising: obtaining a blood, plasma or serum sample from a patient; detecting the levels of at least one diagnostic marker in said sample; wherein the diagnostic marker is selected from diagnostic markers 1-39 and 40-54 of Table 1; and wherein if any diagnostic marker 1-39 is up-regulated, or any diagnostic marker 40-54 is down-regulated, then the patient is at increased risk of carrying a fetus with Down's syndrome.

The detecting step can be any RNA, or protein based detecting step known in the art, but some embodiments are a protein based detecting step, and in others it is an RNA based detecting step.

A method of diagnosing Down's syndrome, the method comprising:
a) obtaining a blood, plasma or serum sample from a patient;
b) purifying proteins from said sample;
c) detecting the levels of one or more diagnostic markers in said purified proteins;
d) wherein said one or more diagnostic markers is selected from diagnostic markers 1-39 and 40-54 of Table 1, and wherein if one or more of diagnostic markers 1-39 is up-regulated, or one or more of diagnostic markers 40-54 is down-regulated, then said patient is at increased risk of carrying a fetus with Down's syndrome; and
e) providing a report of the results obtained in steps c) and d).

A method comprising detecting the levels of at least 4, 5, 6, 7, 8, 9, 10 diagnostic markers.

A method of diagnosing Down's syndrome, the method comprising:
a) obtaining a blood, plasma or serum sample from a patient;
b) purifying RNA from said sample;
c) detecting the levels of RNA encoding one or more diagnostic markers in said purified RNA;
d) wherein said one or more diagnostic markers is selected from diagnostic markers 1-39 and 40-54 of Table 1, and wherein if one or more of diagnostic markers 1-39 is up-regulated, or one or more of diagnostic markers 40-54 is down-regulated, then said patient is at increased risk of carrying a fetus with Down's syndrome; and
e) providing a report of the results obtained in steps c) and d).

The detecting step can be any well-known or future developed detecting step, preferably involving detecting protein or RNA levels. Highly multiplexed assays are possible with e.g., microtitre plates and robotic arms that can be used in laboratory environments, but preferred methods involve arrays of detection antibodies and/or oligonucleotides on a solid format, such that small lab-on-chip and other point of care diagnostic platforms can be used in the method. Such platforms are already commercially available, e.g., from OPKO Diagnostics, formerly Claros Diagnostics, Inc., PalmStat, OJ Bio, SpinDx, XEN BioFluidX, to name a few, and additional platforms are in development, e.g., the McDevitt platform being developed at Rice University, Houston Tex. Another possible platform is the dipstick method, such as is used for pregnancy or HIV testing. However, dipstick technology at this time cannot be greatly multiplexed, and may thus be less desirable.

Protein detection steps are well known, and include 2D gel electrophoresis, 3D gel electrophoresis, Western blots, antibody assays, and sandwich antibody assays. One preferred method is a sandwich or ELISA assay, wherein proteins are captured with a capture antibody, preferably bound to a solid substrate, but possibly also in solution. Then a labeled detection antibody can then be used to provide a detectable signal.

RNA detection steps are well known, and include Northern blots, RT-PCR and other amplification based detection methods. One preferred method is a quantitative reverse transcription to make cDNA, which can then be assayed in any way known in the art, but it typically assayed by hybridization to a labeled probe specific for the diagnostic marker of interest.

Labels can be any known in the art or to be developed. Typically labels include radiolabel, fluorescent label, chemiluminescent label, enzymatic label, immunogenic label, hapten label, magnetic labels, but other detection methods are also possible and include mass detection, detection of changes in electronic properties, chelating dyes, such as EtBR, and the like.

At the completion of the test, a report is provided to the user or patient. Such reports can be in any form, including digital displays on a monitor or cell phone, printouts, and/or links to web-posted data. Preferably, the data is compiled by a computer program or other processor, and a risk evaluation number is provided. If the risk is high, the doctor may follow up with amniocentesis, and chromosome squash to confirm that a trisomy is indeed present.

Risk evaluations can be simple numbers, where only a single marker is detected, or can be fit into a logistic regression type equation that weighs each factor in order to produce a score corresponding to trisomy risk:

trisomy score=a₀+a₁×P₁+a₂×P₂+ . . . +aₙ×Pₙ

where a₁_{_{→}}ₙ are weighing coefficients accounting for the variable importance of each biomarker, and P₁_{_{→}}ₙ are the biomarker levels.

However, such is only one way of calculating risk, and many mathematical methods are possible. Work is ongoing to determine the weighing coefficients and discriminatory value of the various biomarkers, as well as precision (+/−standard error) and bias (95% confidence limits). With such data, we will be able to provide a panel of 3-10 biomarkers that provide both sensitivity and precision, and with only a simple blood test being required. We will also test biomarker levels in urine, to determine if urine could be a suitable sample for at least a subset of the biomarkers, and if so, simple dipstick test kits for home use can also be developed.

When we refer to “obtaining,” “purifying,” “detecting” and “providing”, we include both direct and indirect means therefor. Thus, it is possible, e.g., that a nurse might collect the sample, a separate lab facility perform the remaining steps, and such is to be included within the scope of such verbs.

The term “different expression pattern”, as used throughout this specification, indicates that the expression of a diagnostic marker in the blood or serum of the test subject is different to the normal expression level of that marker i.e., different to the levels found in the blood, plasma or serum of pregnant women whose fetuses do not have Down's syndrome.

The term “an increased amount” or “up-regulated” as used throughout this specification, indicates that the amount of diagnostic marker in the blood or serum of a pregnant woman, is greater than normal i.e. greater than the levels found in the blood, plasma or serum of pregnant women whose fetuses do not have Down's syndrome. In some embodiment, the increased amount is at least 10% higher than that of the normal value. In preferred embodiment, the increased amount is at least 30% higher than that of the normal value, and more preferably 50% higher than that of the normal value.

The term “a decreased amount” or “down-regulated” as used throughout this specification, indicates that the amount of a diagnostic marker in the blood or serum of a pregnant woman is less than normal i.e. less than the levels found in the blood, plasma or serum of pregnant women whose fetuses do not have Down's syndrome. In some embodiment, the decreased amount is at least 10% lower than that of the normal value. In preferred embodiment, the decreased amount is at least 30% lower than that of the normal value, and more preferably 50% lower than that of the normal value.

According to another aspect of the invention, there is provided a method of diagnosing Down's syndrome, the method comprising identifying an increased amount of at least one diagnostic marker in the blood, plasma or serum of a patient compared to the normal expression pattern of the marker, characterized in that the diagnostic marker is selected from markers number 1 to 39 inclusive of those shown in Table 1.

According to another aspect of the invention, there is provided a method of diagnosing Down's syndrome, the method comprising identifying a decreased amount of at least one diagnostic marker in the blood, plasma or serum of a patient compared to the normal expression pattern of the marker, characterized in that the diagnostic marker is selected from markers number 40 to 54 inclusive of those shown in Table 1.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as instructions for use, buffers, wash steps, and the like.

The following abbreviations are used herein:

| ABBREVIATION | TERM |
|---|---|
| PCR | Polymerase chain reaction |
| RT | reverse transcription |
| RNA | Ribonucleic acid |
| DNA | Deoxyribonucleic acid |
| EtBR | Ethidium Bromide |
| DGA | Discriminatory gene analysis |
| CR | Common reference |

### METHODS

### Analysis of the Expression of Genes in Placental Tissue

Gene expression of placental tissue obtained with full ethical consent from women who chose to terminate both normal and Down syndrome fetuses was undertaken. The samples were classified according to the parameters karyotype, gestational age and gender. Among these, the karyotype may adopt the values T21, Normal-N or N, while the samples were either derived from female (F) or male (M) fetal chorionic villi. All samples are from time points near the first-to-second trimester transition and are detailed in Table 2.

TABLE 2
Sample details

| Sample | | | Gestational |
|---|---|---|---|
| Number | Karyotype | Sex | age (week.days) |
| 1 | Normal | M | 12.6 |
| 2 | Normal | M | 12.1 |
| 3 | Normal | M | 12.4 |
| 4 | Normal | M | 12.2 |
| 5 | Normal | M | 13.4 |
| 6 | Normal | M | 12.2 |
| 7 | Normal | M | 13.1 |
| 8 | Normal | M | 12.4 |
| 9 | Normal | M | 12.6 |
| 10 | Normal | F | 13.2 |
| 11 | Normal-N | M | 12.2 |
| 12 | Normal-N | M | 12.3 |
| 13 | Normal-N | M | 13.3 |
| 14 | Normal-N | M | 12.6 |
| 15 | Normal-N | M | 14 |
| 16 | Normal-N | F | 13 |
| 17 | Normal-N | M | 12.6 |
| 18 | Normal-N | M | 12.3 |
| 19 | Normal-N | M | 12.4 |
| 20 | Normal-N | M | 12.6 |
| 21 | T21 | M | 12.5 |
| 22 | T21 | M | 12.4 |
| 23 | T21 | F | 12.2 |
| 24 | T21 | F | 12.6 |
| 25 | T21 | F | 13 |
| 26 | T21 | F | 12.2 |
| 27 | T21 | M | 12.3 |
| 28 | T21 | F | 13.4 |
| 29 | T21 | M | 13.3 |
| 30 | T21 | F | 12.6 |
| 31 | T21 | M | 12.4 |
| 32 | T21 | F | 12.1 |
| 33 | T21 | M | 13.5 |
| 34 | T21 | M | 13.6 |
| 35 | T21 | M | 13 |
| 36 | T21 | F | 12.2 |
| 37 | T21 | M | 12.1 |
| 38 | T21 | F | 12.2 |
| 39 | T21 | F | 13.1 |
| 40 | T21 | F | 12.6 |

40 microarray datasets were generated by single-color hybridization of human RNAs on Agilent Whole Human Genome Oligo Microarrays after T7 RNA amplification. The samples were derived from chorionic villus samples at a gestational age between week 12 and 14. Each sample represents a different donor and fetuses were either male or female. The 40 microarray datasets were subdivided into three karyotype classes (T21, Normal and Normal-N). While Normal denotes fetuses with normal karyotype but a conspicuous maternal serum marker indicative of trisomy 21, Normal-N has both a normal karyotype and normal maternal serum markers. Hence, Normal-N labeled samples were used as control.

Ratios were computed using the RosettaResolver™ Software (Rosetta Inpharmatics). A common reference (CR) was computed by creating an artificial pool of all Normal-N samples. All ratio data were transformed to logarithms to the base 2 logarithms (log2 ratio). In addition, for each ratio the corresponding ‘fold-change’ was computed for a more intuitive understanding of the expression changes.

The unfiltered expression ratios (based on the common reference) of all 40 samples in this analysis were compared in a correlation analysis.

Discriminatory gene analysis (DGA) was undertaken to test each gene for expression differences between the comparison groups. A two-group t-test was performed comparing the groups pairwise, requiring a Bonferroni-corrected p-value of 0.05 or better.

Ratios were computed using the RosettaResolver™ Software (Rosetta Inpharmatics). A common reference (CR) was computed by creating an artificial pool of all Normal-N samples.

### Results

We selected the most highly up-regulated or down- regulated genes, shown in Tables 3 and 4, respectively. Many of these are soluble proteins, which will be found in maternal blood and thus available as diagnostic markers.

TABLE 3
Genes highlyupregulated in Down's Syndrome samples (T21)

| Marker | | | | | |
|---|---|---|---|---|---|
| No | SeqName | Gene description/name | Seqcode | Accession No. | Position |
| 1 | TAS1R1 | taste receptor type 1 | A 23 PI60886 | AL591866 | ChrI |
| | | member 1 | | | |
| 2 | ZNF704 | Zinc finger protein 704 | A 24 P230074 | AK131274 | Chr8 |
| 3 | ATP50 | ATP synthase 0 subunit, | A 23 P143474 | AK222608 | Chr21 |
| | | mitochondrial precursor | | | |
| 4 | AGPAT3 | i-acylglycerol-3-phosphate | A 23 P356466 | AK074300 | Chr21 |
| | | O-acyltransferase 3, | | | |
| | | isoform CRA b | | | |
| 5 | TRIM46 | Tripartite motif protein 46 | A 23 P46222 | AK026882 | ChrI |
| 6 | MPZL2 | Myelin protein zero-like | A 23 PI50379 | BC017774 | Chr 11 |
| | | protein 2 precursor | | | |
| 7 | ATP5J | ATP synthase | A 24 P745670 | AL110183 | Chr21 |
| | | coupling factor 6, | | | |
| | | mitochondrial | | | |
| 8 | MCM3AP | 80 kDa MCM3-associated | A 23 P120744 | AB005543 | Chr 21 |
| | | protein | | | |
| 9 | SMR3A | Submaxillary gland | A 23 P41365 | AC 106884 | Chr4 |
| | | androgen-regulated protein | | | |
| | | 3 homolog A precursor | | | |
| 10 | BC009749 | Human cDNA clone | A 24 P592318 | BC009749 | Chr9 |
| 11 | AAK1 | AP2-associated | A 23 P209826 | AB028971 | Chr2 |
| | | protein kinase 1 | | | |
| 12 | NR1D1 | Orphan nuclear | A 24 P250227 | BC047875 | ChrI7 |
| | | receptor NR1D1 | | | |
| 13 | CSTB | Cystatin-B (Stefin-B) (Liver | A 23 PI54894 | AB083085 | Chr21 |
| | | thiol proteinase inhibitor) | | | |
| 14 | GDNF-002 | glial cell derived | A 24 P376451 | AF053748 | Chr5 |
| | | neurotrophic factor | | | |
| 15 | HSPB9 | Heat-shock protein beta- | A 23 P416212 | AJ302068 | ChrI7 |
| | | 9 (HspB9). | | | |
| 16 | MORC3 | MORC family CW-type | A 23 P325501 | AP000692 | Chr21 |
| | | zinc finger protein 3 | | | |
| 14 | SPINK7 | Serine protease | A 23 P213832 | AF268198 | Chr5 |
| | | inhibitor Kazal-type 7 | | | |
| 18 | RIPK4 | Receptor-interacting | A 24 P125871 | AB047783 | Chr21 |
| | | serine/threonine- | | | |
| | | protein kinase 4 | | | |
| 19 | SOCS1 | Suppressor of cytokine | A 24 P48014 | AB000676 | ChrI6 |
| | | signaling 1 | | | |
| 20 | AL137495 | cDNA clone | A 24 P655646 | AL137495 | Chr4 |
| | | DKFZp434C2331 | | | |
| 21 | DUSP15 | Dual specificity protein | A 23 P154771 | AL160175 | Chr20 |
| | | phosphatase 15 | | | |
| 22 | TNRC18 | Trinucleotide repeat- | A 24 P75245 | U80753 | Chr7 |
| | | containing protein 18 | | | |
| 23 | USP16 | Ubiquitin carboxyl-terminal | A 23 P257911 | AF113219 | Chr21 |
| | | hydrolase 16 | | | |
| 24 | PHOX2A | Paired mesoderm | A 24 P215445 | AF022722 | ChrII |
| | | homeobox protein 2A | | | |
| 25 | STK11 | Serine/threonine- | A 23 PI6483 | U63333 | ChrI9 |
| | | protein kinase 11 | | | |
| 26 | RRBP1 | Ribosome-binding protein 1 | A 32 P28309 | AI916036 | Chr20 |
| 27 | BX090181 | Transcribed locus | A 32 P9518 | BX090181 | |
| | | BX090181 | | | |
| 28 | ATP5J | ATP synthase | A 23 PI54832 | AL110183 | Cnr21 |
| | | coupling factor 6, | | | |
| | | mitochondrial | | | |
| 29 | ZNF488 | Zinc finger protein 488 | A 24 P398210 | BC051323 | ChrIO |
| 30 | C16orf3 | Uncharacterized | A 23 P344515 | AF050080 | ChrI6 |
| | | protein C16orf3 | | | |
| 31 | PCNT | Pericentral (Pericentral | A 23 P57347 | AK024009 | Chr21 |
| | | B) (Kendrin) | | | |
| 32 | E4F1 | E4F transcription factor 1 | A 24 P205100 | NM 004424.3 | ChrI6 |
| 33 | ETS2 | v-ets erythroblastosis | A 23 P257924 | NM 005239 | Chr21 |
| | | virus E26 oncogene | | | |
| | | homolog 2 (avian) | | | |
| 34 | SPIRE2 | spire homolog 2 | A 24 P544996 | AJ422077 | ChrI6 |
| 35 | PWP2 | Periodic tryptophan | A 23 PI02925 | AB001517 | Chr21 |
| | | protein 2 homolog | | | |
| 36 | GART | Glycinamide | A 23 P80098 | AB208785 | Chr21 |
| | | ribonucleotide synthetase | | | |
| 37 | N6AMT1 | HemK methyltransferase | A 23 P80086 | AF139682 | Chr21 |
| | | family member 2 | | | |
| 38 | LOC646960 | Trypsin-like serine protease | A 24 P15182 | NT 005403.17 | Chr21 |
| 39 | RRP1 | RRPI-like protein (Protein | A 23 P80129 | AK223185 | Chr21 |
| | | NNP-1) | | | |

TABLE 4
Genes highly down regulated in Down's syndrome samples (T21)

| Marker | | | | | |
|---|---|---|---|---|---|
| No | SeqName | Gene description/name | Seqcode | Accession No. | Position |
| 40 | IGHV1-46 | Immunoglobulin heavy | A 32 P190951 | J00240 | ChrI4 |
| | | chain V gene segment | | | |
| 41 | CHCHD2 | Coiled-coil-helix-coiled-coil- | A 24 P400376 | AC006970 | Chr7 |
| | | helix domain-containing | | | |
| | | protein 2 | | | |
| 42 | POLK | DNA polymerase kappa | A 23 P386450 | Q9UBT6 | Chr2 |
| 43 | IHPK2 | Inositol hexakisphosphate | A 23 P301133 | Q9UHH9-2 | Chr3 |
| | | kinase 2 | | | |
| 44 | ZNF625 | Zinc finger protein 625 | A 23 P4850 | BC101591 | ChrI9 |
| 45 | LEPR | leptin receptor | A 23 P161135 | | |
| 46 | BVES | Blood vessel epicardial | A 23 P502783 | AF124512 | Chr6 |
| | | substance | | | |
| 47 | C13orf30 | Uncharacterized protein | A 32 P332551 | AK098238 | ChrI3 |
| | | C13orf30 | | | |
| 48 | ING5 | Inhibitor of growth protein 5 | A 23 P124202 | AK128322 | Chr2 |
| 49 | ZC3H5/UNK | Zinc finger CCCH domain- | A 32 P514790 | AB051540 | ChrI7 |
| | | containing protein 5. | | | |
| 50 | RHO | Rhodopsin (Opsin-2). | A 23 P57950 | AB065668 | Chr3 |
| 51 | DMC1 | Meiotic recombination | A 23 P361381 | AL022320 | Chr22 |
| | | protein DMC1/LIM15 | | | |
| | | homolog | | | |
| 52 | CD1B | CD Ib molecule | A 23 P351844 | NM 001764.2 | Chr 1 |
| 53 | DKFZp761E198 | Putative uncharacterized | A 23 P24424 | AL834269 | ChrII |
| | | protein DKFZp761E198 | | | |
| 54 | DNAH8 | Ciliary dynein heavy chain 8 | A 23 P145159 | AF527621 | Chr 6 |

From the results shown in Tables 3 and 4, it's clearly shown that the up-regulation of markers 1-39 or the down-regulation of markers 40-54 bears a strong co-relation with Down's syndrome. It is expected that in a blood, plasma or serum sample from a pregnant woman will also contain at least 1, 2, 3, 4, 6, 8, 10 or more of these markers for screening Down's Syndrome of the fetus. Therefore, non-invasive method of obtaining samples from a pregnant woman is possible for Down's syndrome screening. These markers have not been reported as markers for screening Down's syndrome.

1. A method of diagnosing Down's syndrome, the method comprising:
a) obtaining a blood, plasma, or serum sample from a patient;
b) detecting the levels of at least one diagnostic marker in said sample;
c) wherein said diagnostic marker is selected from diagnostic markers 1-39 of Table 3 and 40-54 of Table 4; and
d) wherein if diagnostic marker 1-39 is up-regulated, or diagnostic marker 40-54 is down-regulated, then said patient is at increased risk of carrying a fetus with Down's syndrome.

2. The method of claim 1, wherein said detecting step is a protein based detecting step.

3. The method of claim 1, wherein said detecting step is a RNA based detecting step.

4. A method of diagnosing Down's syndrome, the method comprising:
a) obtaining a blood, plasma or serum sample from a patient;
b) purifying proteins from said sample;
c) detecting the levels of one or more diagnostic markers in said purified proteins;
d) wherein said one or more diagnostic markers is selected from diagnostic markers 1-39 of Table 3 and 40-54 of Table 4, and wherein if one or more of diagnostic markers 1-39 is up-regulated, or one or more of diagnostic markers 40-54 is down-regulated, then said patient is at increased risk of carrying a fetus with Down's syndrome; and
e) providing a report of the results obtained in steps c) and d).

5. The method of claim 4, wherein said detecting step c) is an antibody based detecting step.

6. The method of claim 4, wherein said detecting step c) is a two antibody based detecting step using a capture antibody and a labeled detection antibody.

7. The method of claim 5, wherein said antibody is labeled with a fluorescent dye.

8. The method of claim 6, wherein said labeled detection antibody is labeled with a fluorescent dye.

9. The method of claim 4, said method comprising detecting the levels of at least three diagnostic markers.

10. The method of claim 4, said method comprising detecting the levels of at least 4,5,6,7,8,9, or 10 diagnostic markers.

11. A method of diagnosing Down's syndrome, the method comprising:
a) obtaining a blood, plasma or serum sample from a patient;
b) purifying RNA from said sample;
c) detecting the levels of RNA encoding one or more diagnostic markers in said purified RNA;
d) wherein said one or more diagnostic markers is selected from diagnostic markers 1-39 and 40-54 of Tables 3-4, and wherein if one or more of diagnostic markers 1-39 is up-regulated, or one or more of diagnostic markers 40-54 is down-regulated, then said patient is at increased risk of carrying a fetus with Down's syndrome; and
e) providing a report of the results obtained in steps c) and d).

12. The method of claim 11, wherein said detecting step c) further comprising RT-PCR amplification of said purified RNA to make amplified DNA, followed by hybridization of said amplified DNA to probes specific for said one or more diagnostic markers.

13. The method of claim 11, said method comprising detecting the levels of at least three diagnostic markers.

14. The method of claim 11, said method comprising detecting the levels of at least 4,5,6,7,8,9, or 10 diagnostic markers.

15. A method of diagnosing Down's syndrome, the method comprising:
a) obtaining a sample from a patient;
b) detecting the levels of at least one diagnostic marker in said sample;
c) wherein said diagnostic marker is selected from diagnostic markers 1-39 of Table 3 and 40-54 of Table 4;
d) wherein if diagnostic marker 1-39 is up-regulated, or diagnostic marker 40-54 is down-regulated, then said patient is at increased risk of carrying a fetus with Down's syndrome; and
e) providing a report of the results obtained in steps c) and d).

16. The method of claim 15, wherein said detecting step is a protein based detecting step.

17. The method of claim 15, wherein said detecting step is a RNA based detecting step.

18. The method of claim 15, wherein said sample is a blood sample.
